(19) United States
(12) Patent Application Publication
(10) Pub. No.: US 2026/0318831 A1
(43) Pub. Date: Oct. 15, 2026

(54) A CONSTANT CONVERSION RATE OF ENDOLYSOSOMES REVEALED BY A PH-SENSITIVE FLUORESCENT PROBE

(71) Applicant: University of Cincinnati, Cincinnati, OH (US)

(72) Inventors: Yujie Sun, Mason, OH (US); Jiajie Diao, Lebanon, OH (US)

(73) Assignee: University of Cincinnati, Cincinnati, OH (US)

(21) Appl. No.: 19/482,487

(22) PCT Filed: May 8, 2024
(86) PCT No.: PCT/US2024/028334

§ 371 Date: Nov. 7, 2025

Related U.S. Application Data
(60) Provisional application No. 63/466,129, filed on May 12, 2023.

(51) Int. Cl. G01N 21/64; C09B 23/14

(52) U.S. Cl. CPC G01N 21/6428; C09B 23/145; G01N 21/6458; G01N 2021/6439

(57) ABSTRACT

The present disclosure concerns fluorescent probes of pyridine, quinolinium and piperazine covalently linked as a small molecule probe. The probes can be administered to cells and are readily uptaken thereby. The probes locate particularly into endolysosomes. The probes provide two types of fluorescence with pH-sensitive photophysical properties, which in turn allows for the observation and analysis of changes occurring during varying stages of interest. The dual fluorescence provides for simultaneous monitoring of two fluorescence signals in endolysosomes at different stages of interest and minimizes the interference from various microenvironments.

### CROSS REFERENCE TO RELATED APPLICATION

This application claims priority to U.S. Provisional Application Ser. No. 63/466,129, filed May 12, 2023, the entire contents of which are incorporated herein by reference.

### STATEMENT REGARDING FEDERALLY SPONSORED RESEARCH OR DEVELOPMENT

This invention was made with government support under R35GM128837 awarded by National Institutes of Health and CHE1955358 awarded by the National Science Foundation. The government has certain rights in the invention.

### TECHNICAL FIELD

The present disclosure concerns fluorescent probes and their methods of use in imaging.

### BACKGROUND

Autophagy is a conserved lysosomal degradation pathway, which maintains cellular homeostasis and health by removing unnecessary or dysfunctional substances. Endolysosomes, including early endosomes, late endosomes, and lysosomes, are critical for autophagosome biogenesis during the process of autophagy and hence imaging the subcellular dynamics of endolysosomes with a high spatial-temporal resolution will significantly benefit our understanding of autophagy and will also provide the opportunity to develop new therapeutic strategies for lysosome-related neurological disorders, such as Alzheimer's disease. It has been determined that the number of lysosomes increases dramatically during autophagy, while the mechanism behind it remains unknown. This triggers a search for the reason behind the dramatic increase in lysosomes during autophagy. Therefore, it was of interest to develop an appropriate probe to study the endolysosome dynamics by illustrating the different stages of endolysosomes with different fluorescence signals.

Designing high-performance fluorescent probes suitable for in-situ real-time bioimaging faces challenges, including specific targeting ability, high sensitivity, long-term photostability, high signal-to-noise ratio, and low toxicity. In the past few years, scientists have developed a series of cell-permeable organic fluorophores for lysosomes with various colors that exhibit favorable bioimaging properties. To further observe the dynamics of lysosomes, the general microenvironment changes in lysosomes can be used as versatile and effective markers, such as viscosity and polarity. Since the local pH decreases substantially from early endosome (pH 6.3) to late endosome (pH 5.5) and lysosome (pH~4.7) while the extracellular medium, cytosol, and nucleus all maintain a nearly neutral pH 7.2-7.4, such a significant pH difference of endolysosomes at different stages can be utilized in designing fluorescent probes for tracking their dynamics. Recently, real-time imaging of cellular uptake via endocytosis has been successfully achieved by using a pH-sensitive lysosomal fluorophore in which protonation occurs on a neutral phenol moiety. However, most reported pH-sensitive probe designs only result in “turn-on” probes at low pH, which are nearly non-fluorescent at high pH, or vice versa. In order to precisely track the dynamics of endolysosomes via fluorescence microscopy techniques, it is necessary to design novel probes with pH-dependent evolution and large Stokes shifts that can be imaged simultaneously in multiple channels with low background noise.

Even though fluorescence microscopy has demonstrated its value in the visual analysis of substances within cells as well as living organisms, the practical resolution limit of optical microscopes (roughly 200 nm) restricts its applicability. Recently, several novel high-resolution microscopes that can break diffraction limits have been invented for cell imaging to digitize and observe the dynamic changes and interactions of organelles. LIGHTNING is a Leica Microsystems ultra-high-resolution confocal platform, which is based on adaptive computational deconvolution and has overcome the resolution down to 120 nm with simultaneous multicolor imaging. With the LIGHTING mode, the details of living samples can be efficiently tracked with fast acquisition speed, and it can greatly prevent photodamage caused by using high-energy laser sources.

### SUMMARY

The present disclosure concerns fluorescent probes and their methods of use in imaging. In some aspects, the fluorescent probe is a small molecule that is taken up into endolysosomes and helps identify different characteristics during the lifespan thereof. In some aspects, the fluorescent probes fluoresce under two or more different intensities.

A 1^{st} aspect of the present disclosure, either alone or in combination with any other aspect set forth herein, concerns a fluorescent probe comprising a molecule comprised of a quinolinium, a pyridine, and a piperazine linked together.

A 2^{nd} aspect of the present disclosure, either alone or in combination with any other aspect set forth herein, concerns the fluorescent probe of the 1^{st} aspect, further comprising a phenyl and a C═C double bond linking the quinolinium and the piperazine.

A 3^{rd} aspect of the present disclosure, either alone or in combination with any other aspect set forth herein, concerns the fluorescent probe of the 1^{st} or 2^{nd} aspect, wherein the piperazine is methylated.

A 4^{th} aspect of the present disclosure, either alone or in combination with any other aspect set forth herein, concerns the fluorescent probe of the 1^{st}, 2^{nd}, or 3^{rd} aspect, wherein the molecule comprises the structure of formula I:

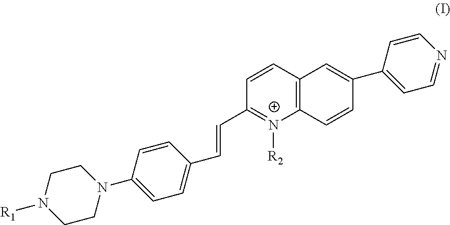

wherein R₁ and R₂ are independently an alkyl of C₁-C₈ in length and wherein the pyridine is optionally protonated.

A 5^{th} aspect of the present disclosure, either alone or in combination with any other aspect set forth herein, concerns the fluorescent probe of the 1^{st} aspect, wherein the molecule comprises the structure of formula II:

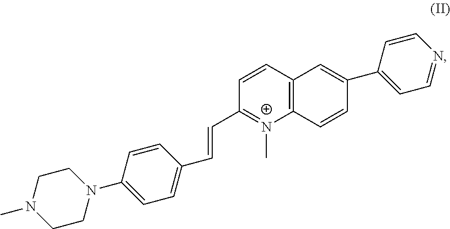 [SMILES: CN1CCN(c2ccc(/C=C/c3ccc4cc(-c5ccncc5)ccc4[n+]3C)cc2)CC1]

wherein the pyridine is optionally protonated.

A 6^{th} aspect of the present disclosure, either alone or in combination with any other aspect set forth herein, concerns the fluorescent probe of the 1^{st}, 3^{rd}, or 5^{th} aspect, further comprising a halide and wherein the fluorescent probe is a salt.

A 7^{th} aspect of the present disclosure, either alone or in combination with any other aspect set forth herein, concerns a method for monitoring endolysosomes in a cell, comprising administering the fluorescent probe of the 1^{st} aspect to a cell.

An 8^{th} aspect of the present disclosure, either alone or in combination with any other aspect set forth herein, concerns the method of the 7^{th} aspect, further comprising viewing the cell at at least one time point and optionally capturing an image of the cell.

A 9^{th} aspect of the present disclosure, either alone or in combination with any other aspect set forth herein, concerns the method of the 8^{th} aspect, wherein the cell is viewed through a green channel, a red channel, or both.

A 10^{th} aspect of the present disclosure, either alone or in combination with any other aspect set forth herein, concerns the fluorescent probe of the 7^{th}, 8^{th}, or 9^{th} aspect, wherein the cell is treated with a further compound at a time point prior to, simultaneously, or after administering the probe to the cell.

An 11^{th} aspect of the present disclosure, either alone or in combination with any other aspect set forth herein, concerns a fluorescent probe comprised of a molecule, wherein the molecule comprises the structure of formula II:

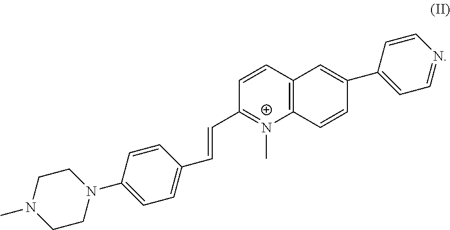 [SMILES: CN1CCN(c2ccc(/C=C/c3ccc4cc(-c5ccncc5)ccc4[n+]3C)cc2)CC1]

A 12^{th} aspect of the present disclosure, either alone or in combination with any other aspect set forth herein, concerns the fluorescent probe of the 11^{th} aspect, wherein the pyridine is optionally protonated.

A 13^{th} aspect of the present disclosure, either alone or in combination with any other aspect set forth herein, concerns the fluorescent probe of the 11^{th} or 12^{th} aspect, further comprising a halide and wherein the fluorescent probe is a salt.

A 14^{th} aspect of the present disclosure, either alone or in combination with any other aspect set forth herein, concerns the fluorescent probe of the 13^{th} aspect, wherein the halide is iodide.

A 15^{th} aspect of the present disclosure, either alone or in combination with any other aspect set forth herein, concerns a method for monitoring endolysosomes in a cell, comprising administering the fluorescent probe of the 11^{th} aspect to a cell.

A 16^{th} aspect of the present disclosure, either alone or in combination with any other aspect set forth herein, concerns the method of the 15^{th} aspect, further comprising viewing the cell at at least one time point and optionally capturing an image of the cell.

A 17^{th} aspect of the present disclosure, either alone or in combination with any other aspect set forth herein, concerns the method of the 16^{th} aspect, wherein the cell is viewed through a green channel, a red channel, or both.

An 18^{th} aspect of the present disclosure, either alone or in combination with any other aspect set forth herein, concerns the fluorescent probe of the 15^{th}, 16^{th}, or 17^{th} aspect, wherein the cell is treated with a further compound at a time point prior to, simultaneously, or after administering the probe to the cell.

### BRIEF DESCRIPTION OF THE DRAWINGS

FIGS. 1A-1B depict the schematic of design and synthesis of PyQPMe. FIG. 1A. Proposed design of a pH-dependent probe to track the endolysosome dynamics. FIG. 1B. Synthetic scheme of the designed fluorescent probe PyQPMe. (i) Pd(PPh₃)₄. K₂CO₃, (Bu)₄NBr, toluene, EtOH, H₂O, N₂, 100° C.; (ii) CH₃I, EtOH or CH₃CN, 80° C.; (iii) K₂CO₃, DMF, 100° C.; (iv) anhydrous EtOH, N₂, 80° C.

FIGS. 2A-2D depict the pH-dependent properties of PyQPMe and computational calculations. FIG. 2A. Absorption spectra of PyQPMe in buffer solutions (pH 4.0-8.0). FIG. 2B. Emission spectra of PyQPMe in buffer solutions with different pH excited at 405 nm. FIG. 2C. I_{645 nm}/I_{500 nm} emission intensity ratio of PyQPMe in buffer solutions (pH 4.0-8.0). FIG. 2D. Calculated HOMOs, LUMOs, and EDDMs of the 1^{st} singlet excited states (S₁) of PyQPMe and protonated PyQPMe. Isovalue=0.04 for plotting HOMOs, LUMOs, and EDDMs.

FIGS. 3A-3E depict the characterization of PyQPMe in living cells. FIG. 3A. Confocal imaging of PyQPMe co-stained with LTDR in HeLa cells. FIG. 3B, 3C. Zoom-in images of white rectangles in FIG. 3A. FIG. 3D. Fluorescence intensity ratio of PyQPMe-stained early endosomes and late endosomes/lysosomes. FIG. 3E. PCC value of PyQPMe co-stained with LTDR. Green channel, λₑₓ=405 nm, λₑₘ=470-530 nm; Red channel, λₑₓ=405 nim, λₑₘ=610-680 nm; Deep red channel, λₑₓ=640 nm, λₑₘ=690-740 nm.

FIGS. 4A-4G depict the characterization of imaging properties of PyQPMe-stained endolysosomes. FIG. 4A. Confocal image of PyQPMe-stained endolysosomes and fluorescence intensity profile (FIG. 4B) of the write line in FIG. 4A. FIG. 4C. Confocal image of PyQPMe-stained late endosomes/lysosomes and fluorescence intensity profile (FIG. 4D) of the write line in FIG. 4C. FIG. 4E. Confocal images of HeLa cells labeled with LTR and PyQPMe and 3D fluorescence intensity plots of rectangles of integration. FIG. 4F. Continuous imaging of HeLa cells labeled with LTR or PyQPMe at 100% laser intensity for photobleaching, and each scan time was 10 s, images were captured under a red channel. FIG. 4G. Normalized fluorescence intensity of LTR and PyQPMe in FIG. 4F.

FIGS. 5A-5H depict the characterization of dynamics of PyQPMe-stained endolysosomes. FIG. 5A. Confocal images of HeLa cells stained with PyQPMe following treatment with or without Wortmannin for 24 h. FIG. 5B. Schematic illustration of the effect of Wortmannin which inhibits the conversion from early endosomes into late endosomes. FIG. 5C. Quantification of total endolysosomes and late endosomes/lysosomes in PyQPMe-stained HeLa cells with or without Wortmannin treatment. FIG. 5D. The ratio of the number of late endosomes/lysosomes to total endolysosomes in PyQPMe-stained HeLa cells treated with or without Wortmannin. FIG. 5E. Confocal images of HeLa cells stained with PyQPMe for 10 min and 60 min. FIG. 5F. The mean red puncta fluorescence intensity of PyQPMe-stained HeLa cells for 10 min and 60 min incubation. FIG. 5G. Quantification of total endolysosomes and late endosomes/lysosomes in PyQPMe-stained HeLa cells for 10 min and 60 min. FIG. 5H. The ratio of the number of late endosomes/lysosomes to total endolysosomes in PyQPMe-stained HeLa cells for 10 min and 60 min. Data are given as M±SEM (n=10 independent samples from 10 cells for each statistical analysis); the statistical differences were analyzed by Student's t-test, p<0.05 is considered to statistical significant (** p≤0.01; *** p≤0.001; **** p≤0.0001; ns p>0.05).

FIGS. 6A-6H depict the dynamics of PyQPMe-stained endolysosomes during autophagy. FIG. 6A. Confocal images of HeLa cells incubated with PyQPMe following treatment with or without CCCP for 24 h. FIG. 6B. Quantification of total endolysosomes and late endosomes/lysosomes in PyQPMe-stained HeLa cells with or without CCCP treatment. FIG. 6C. The ratio of the number of late endosomes/lysosomes to total endolysosomes in PyQPMe-stained HeLa cells treated with or without CCCP. FIG. 6D. Quantification of ratio of red to green intensity in PyQPMe-stained HeLa cells without CCCP treatment. FIG. 6E. Quantification of ratio of red to green intensity in PyQPMe-stained HeLa cells with CCCP treatment for 24 h. FIG. 6F. Confocal images of HeLa cells incubated with PyQPMe following treatment with or without rapamycin for 24 h. FIG. 6G. Quantification of total endolysosomes and late endosomes/lysosomes in PyQPMe-stained HeLa cells with or without rapamycin treatment. FIG. 6H. The ratio of the number of late endosomes/lysosomes to total endolysosomes in PyQPMe-stained HeLa cells treated with or without rapamycin. Data are given as M±SEM (n=10 independent samples from 10 cells for each statistical analysis); the statistical differences were analyzed by Student's t-test, p<0.05 is considered to statistical significant (**** p≤0.0001; ns p>0.05).

FIG. 7 shows calculated molecular orbitals of PyQPMe in 1120.

FIG. 8 shows calculated singlet electron transitions (f>0.01) of PyQPMe in H₂O.

FIG. 9 shows calculated molecular orbitals of protonated PyQPMe in H₂O.

FIG. 10 shows calculated singlet electron transitions (f>0.01) of protonated PyQPMe in H₂O.

### DETAILED DESCRIPTION

Endolysosome dynamics play an important role in autophagosome biogenesis. Hence, imaging the subcellular dynamics of endolysosomes using high-resolution fluorescent imaging techniques would deepen our understanding of autophagy and benefit the development of pharmaceuticals against endosome-related diseases. Taking advantage of the intramolecular charge transfer mechanism, herein we report a cationic quinolinium-based fluorescent probe (PyQPMe) that exhibits excellent pH-sensitive fluorescence in endolysosomes at different stages of interest. A systematic photophysical and computational study on PyQPMe was carried out to rationalize its highly pH-dependent absorption and emission spectra. The large Stokes shift and strong fluorescence intensity of PyQPMe can effectively reduce the background noise caused by excitation light and microenvironments and provide a high signal-to-noise ratio for high-resolution imaging of endolysosomes. By applying PyQPMe as a small molecular probe in live cells, a constant conversion rate from early endosomes to late endosomes/lysosomes during autophagy at the submicron level is revealed.

The present disclosure concerns fluorescent probes that allow for the detection and/or observation of changes to the microenvironment of an endosome. In some aspects, the fluorescent probe is a small-molecule cationic quinolinium-based fluorescent probe. In some aspects, the fluorescent probe can produce two or more fluorescent signals in an endosome to allow for monitoring different stages of interest of an endosome.

Herein, the present disclosure provides a cationic quinolinium-based fluorescent probe, referred to as “PyQPMe.” In some aspects, the probe includes a pyridine (Py) (or derivative thereof), a quinolinium (Q) (or derivative thereof), and a piperazine (P) (or derivative thereof). In some aspects, the piperazine and/or the quinolinium is methylated (Me). While referred to as the individual parts of the probe, it will be appreciated that each subunit or part within the probe is linked to the other subunits or parts, such as through a covalent bond, thereby providing a single molecule.

In some aspects, a linker or intermediate subunit(s) may be introduced between the listed subunits. For example, PyQP refers to an assembled compound of covalently bound pyridine (Py) (or derivative thereof), quinolinium (Q) (or derivative thereof), and piperazine (P) (or derivative thereof). In some aspects, the quinolinium is protonated. “Derivative” may include one or more substitutions, such as with a halide or an alkyl about the base structure of the named compound. In some aspects, the piperazine is protonated. In some aspects, the quinolinium and the piperazine are protonated. In some aspects, the quinolinium is methylated or appended with a methyl group (Me). In some aspects, the piperazine is methylated or appended with a methyl group. In some aspects, a linker (L) may connect or link the piperazine and the quinolinium. In some aspects, the compound may be of the structure: P-Lₙ-Q-Py and/or Me-Py-Lₙ-Q-P (n being a value of 0 or 1). In some aspects, an interim benzaldehyde can be used to link, providing a phenyl spacer between the piperazine and a C═C double bond to the quinolinium. In some aspects, a styrene moiety may link the quinolinium and piperazine. In some aspects, a double bond can form between a methyl quinolinium and the aldehyde of the linker. An exemplary structure of one aspect of the fluorescent probe is set forth in I:

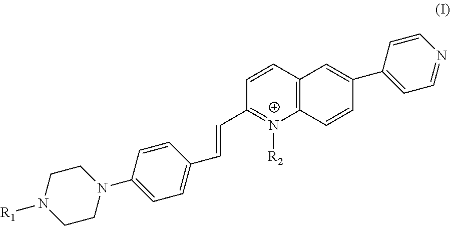

wherein R₁ and R₂ are independently an alkyl of C₁-C₈ in length. In some aspects, R₁ and/or R₂ are methyl, such as that set forth in II:

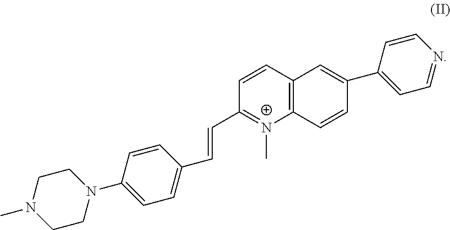 [SMILES: CN1CCN(c2ccc(/C=C/c3ccc4cc(-c5ccncc5)ccc4[n+]3C)cc2)CC1]

In some aspects, the probe may be a salt thereof, such as a chloride, iodide, fluoride, bromide or similar negatively charged anion coupled thereto. In some aspects, the salt is an iodide salt, as set forth in III:

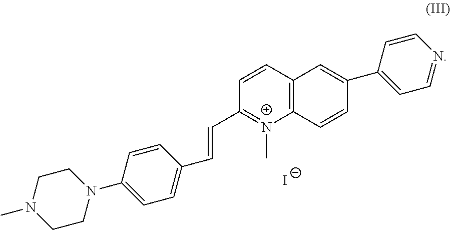 [SMILES: CN1CCN(c2ccc(/C=C/c3ccc4cc(-c5ccncc5)ccc4[n+]3C)cc2)CC1 ; [I-]]

The probes of the present disclosure were designed out of desire to develop a fluorescent probe that includes both electron-donating (e.g., piperazine) and electron-withdrawing (e.g., cationic quinolinium moiety) units connected by a C═C double bond, in part inspired by the rich photophysical properties resulting from intramolecular charge transfer in organic fluorophores. Piperazine is selected as an ideal electron-donating group because it is also a good targeting group towards endolysosomes.³⁸ Furthermore, intending to introduce pH-dependent optical responses for tracking the endolysosome dynamics, installing a pyridyl substituent is an attractive option because the pKa of pyridine is 5.23, perfectly falling within the pH range from early endosomes to lysosomes. Hence, herein is presented a fluorescent probe with pH sensitivity towards endolysosomes, which can be employed as a convenient tool to investigate the endolysosome dynamics in real time by using high-resolution imaging techniques with high spatial resolution and low background noise.

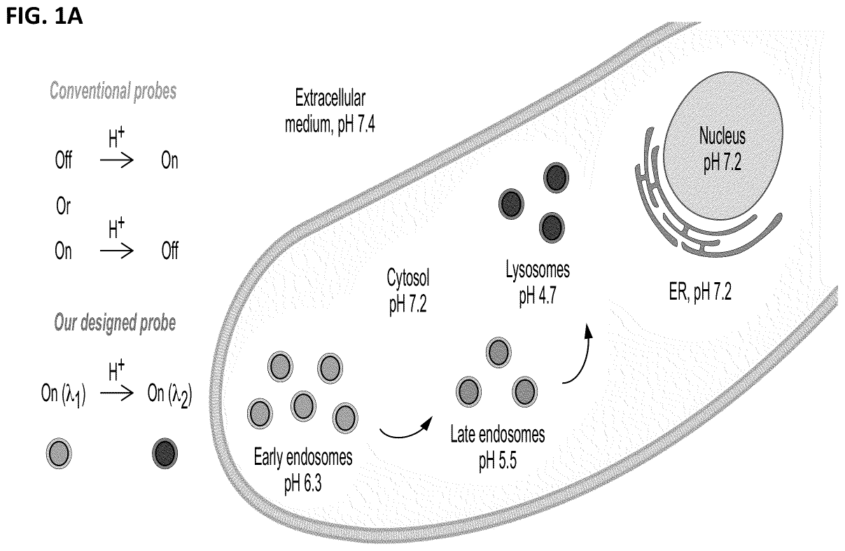

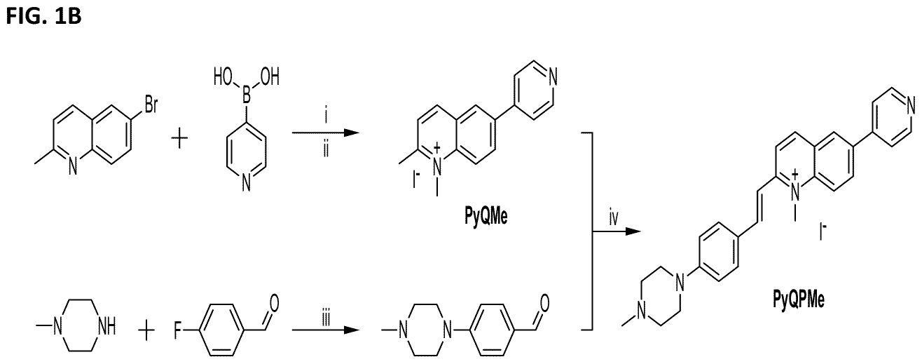

The synthetic scheme of PyQPMe is presented in FIG. 1B, wherein the initial step is the Suzuki coupling between 6-bromo-2-methylquinoline and pyridin-4-ylboronic acid, both of which are commercially available. Subsequent methylation at the quinoline N resulted in the intermediate product PyQMe with an overall yield above 50%. The reaction between 1-methylpiperazine and p-fluorobenzaldehyde under alkaline condition led to the formation of 4-(4-methylpiperazin-1-yl) benzaldehyde in a nearly quantitative yield. A final Knoevenagel condensation between PyQMe and 4-(4-methylpiperazin-1-yl) benzaldehyde realized the synthesis of PyQPMe. The experimental details and characterization for the syntheses of PyQPMe and relevant synthetic intermediates are included in the Examples herein. As set forth herein, the terminal pyridine group of PyQPMe is gradually protonated in cells through the occurrence of endocytosis which results in a red shift of fluorescence, as seen in IV:

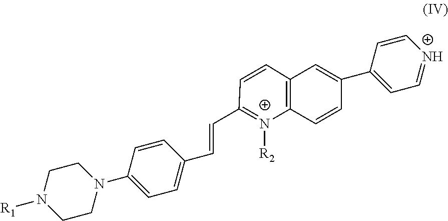

wherein R₁ and R₂ are independently an alkyl of C₁-C₈ in length. In some aspects, R₁ and/or R₂ are methyl, such as that set forth in V:

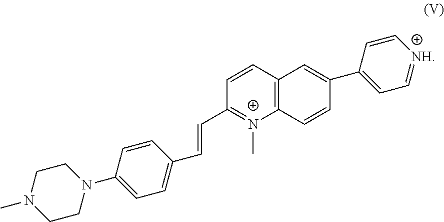

Due to different pH values of endolysosomes at different stages, PyQPMe stains total endolysosomes and late endosomes/lysosomes with green and red fluorescence, respectively.

### Methods

In some aspects, the present disclosure includes methods of using the probes as set forth herein. In some aspects, the methods include administering the fluorescent probes as described herein to a cell. In some aspects, the cell is in vitro. In some aspects, the cell may be in vivo and/or ex situ from that of a subject, such as a mammalian subject. The group and attributes of mammals are well understood in the art and include humans, rodents, bovine, ovine, canine, feline, and similar species/orders. As described herein, administering the probes to cells leads to uptake within endolysosomes of the cell as the probe can readily penetrate or cross into the cytoplasm of the cell where it is then contained or captured for the purpose of phagocytosis to remove the probe or break the probe down. It will be understood that the probes of the present disclosure may be administered as a solution, wherein the probe is soluble and/or suspended in the solution. It will also be understood that a solution may include vehicles, carriers, excipients, etc. as are understood in the art. The probes may also be administered together or independently at different time point(s) with other compounds of interest. For example, one may be testing or observing the effect of a particular pharmaceutical or agent on how such can affect cellular systems. The administration thereof with the probes of the present disclosure allows for the observation of the effects on endolysosome and/or overall phagocytosis within the cell.

The structure of the quinolinium-based fluorescent probes disclosed herein allows for cells to readily uptake the probe when administered. In some aspects, the methods include monitoring endolysosomes through the presence of the probes as set forth herein being located therein or in proximity thereto, such as within a cell. In some aspects, the probe is subject to endocytosis within cells, allowing the probes to effectively stain or fluoresce in endolysosomes. The fluorescent probes, including PyQPMe, allow one to simultaneously monitor two fluorescence signals in endolysosomes at different stages of interest and minimize the interference from various microenvironments; and its large Stokes shift and strong fluorescence intensity properties endow a high signal-to-noise ratio for high-resolution imaging (FIG. 1A). By utilizing this probe for imaging, the dynamics of endolysosomes was further digitized and characterized, which revealed a constant conversion rate from early endosomes to late endosomes/lysosomes during autophagy.

In some aspects, the methods therefore may include imaging the cell(s) once the probe has been taken up therein. Such may also include monitoring the cells over a time course or obtaining multiple image thereof of varying time points. Such may also include viewing the cells under a particular channel or under multiple channels to obtain images of the fluorescent probe in situ within the cell and/or an endolysosome. In some aspects, the methods may include measuring or quantifying fluorescence intensity, such as the brightness under a particular channel, such as a green channel (emission range of 470-530 nm) and/or a red channel (emission range of 610-680 nm). The methods may then include obtaining overlay images collected from different channels. The methods may include preparing a time-lapse or film showing changes in fluorescence over time.

pH-Dependent Properties of PyQPMe and Computational Calculations

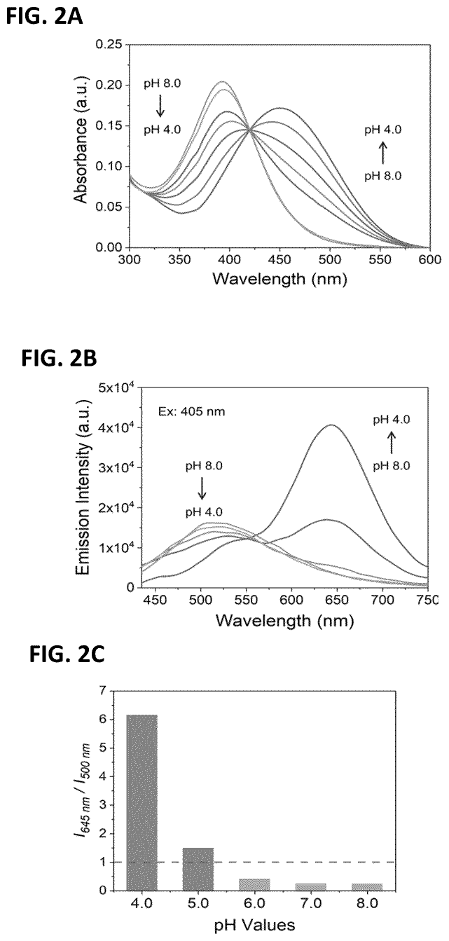

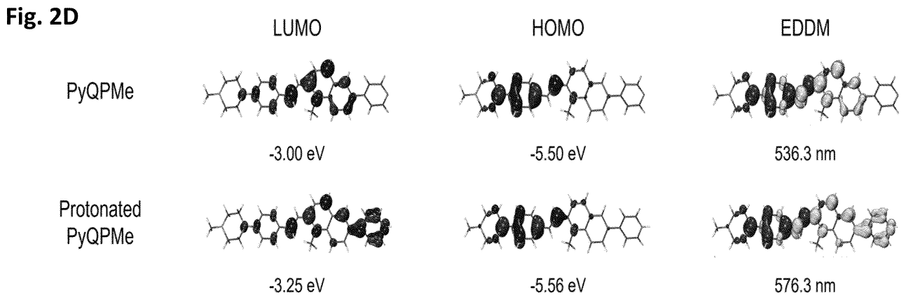

In some aspects, the fluorescence of the probe changes as the pH of an endolysosome changes, such as between a pH range of about 8.0 to about 2.0. With the designed probe PyQPMe in hand, its UV-visible absorption was screened in aqueous buffers with a pH range of 8.0 to 4.0. PyQPMe exhibited substantial spectral evolution along with the pH change, as presented in FIG. 2A. Under the neutral condition, PyQPMe has an absorption maximum at 394 nm (ε=34,147 M⁻¹cm⁻¹), which readily decreases when the pH is below 6. Simultaneously, a new absorption maximum at 447 nm (ε=27,264 M⁻¹ cm⁻¹) rises with its tail exceeding 600 nm in pH 4.0 buffer solution. The absorption intensity evolution at 394 and 447 nm along with the decrease of pH from 8.0 to 4.0 faultlessly elaborate an apparent pKa value of ~5 for PyQPMe. In the meantime, PyQPMe remains fluorescent within the entire pH window of 8.0 to 4.0. Under 405 nm excitation, PyQPMe showed fluorescence with a maximum peak at 510 nm in the neutral solution (FIG. 2B). When the pH decreased from 8.0 to 4.0, the PyQPMe probe displayed a 140 nm red shift with a new emission peak at 650 nm. The Stokes shift of PyQPMe in acid solution is greater than 200 nm, which is due to the extended π-conjugation and enhanced intramolecular charge transfer induced by the protonation of its terminal pyridine group. The quantum yield of PyQPMe was also enhanced to 0.23% in pH 4.0 buffer solution. Moreover, the pH titration curve illustrated a decent linear relationship by plotting the emission intensity ratio of I_{510 nm}=/I_{650 nm} against pH values. In addition, the fluorescence intensities of PyQPMe used for different imaging channels of the microscope were compared, where the wavelengths of 500 nm and 645 nm represent the green and red channels, respectively. When the buffer solution is much more acidic, the fluorescence intensity ratio of I_{645 nm}/I_{500 nm} is higher than 1 (FIG. 2C), indicating red color dominates the fluorescence of PyQPMe. Conversely, with increasing the pH value, green fluorescence light is becoming the predominant color for imaging. This exceptional color evolution property of PyQPMe indicates that it is an ideal pH-sensitive probe for the dual-channel mode of high-resolution imaging.

Next, the effect of potential biochemical species on the fluorescence intensity of PyQPMe in both pH 3.0 and 8.2 buffer solutions was investigated. Various chemical substances, such as cations (K⁺, Na⁺, Mg²⁺, Zn²⁺, Fe²⁺, Fe³⁺, Ag⁺, Ni²⁺), anions (F⁻, Cl⁻, Br⁻, I⁻, SCN⁻, NO₃⁻, HCO₃⁻, SO₄²⁻, HSO₃⁻, Ac⁻), reactive oxygen species (H₂O₂), and reactive sulfur species (Cys, Hcy, GSH) were added to the PyQPMe solution; none of these interfering substances could quench the emission intensity in either pH 3.0 or 8.2 solutions and the protonated PyQPMe exhibited strong fluorescence during the whole process. Fluorescence spectra of PyQPMe in various solvents with different polarities were also obtained. PyQPMe only exhibited significant emission intensity in low pH solution, further confirming that polarity has no effect on the fluorescence of PyQPMe. In addition, as a pH probe, the emission spectra of PyQPMe show excellent reversibility between pH 8.5 and pH 3.0, which is attributed to the protonation/deprotonation of its terminal pyridine group. Furthermore, the photostability of PyQPMe was investigated under different pH conditions. The fluorescence intensity exhibited negligible change after 300 s irradiation in neutral and acidic solutions, confirming its superior photostability. Taken together, the above photophysical results identify that PyQPMe is a pH-sensitive probe, well suited for studying endolysosome dynamics via high-resolution imaging techniques.

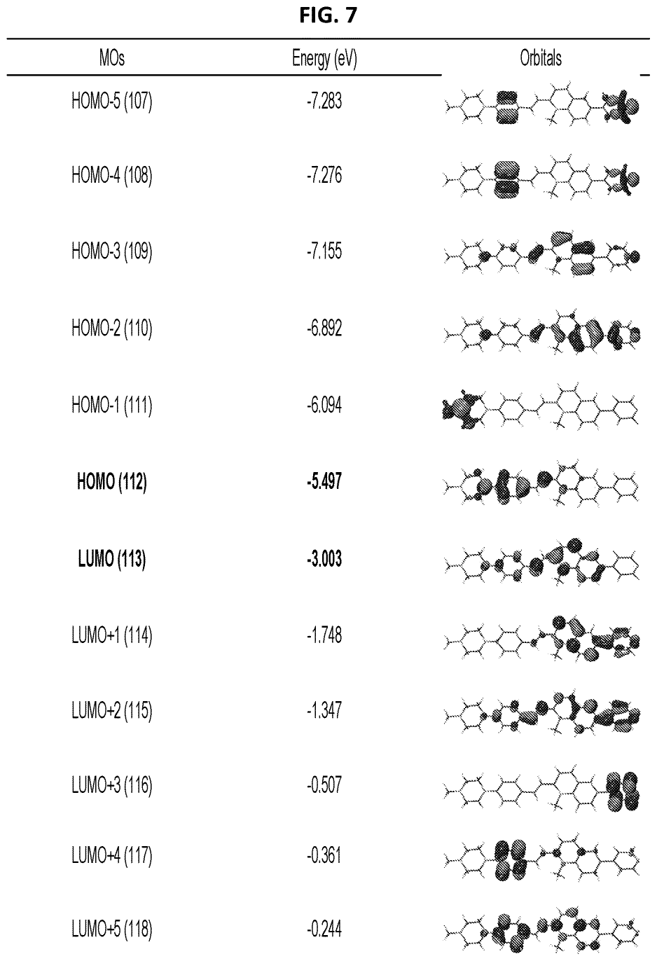

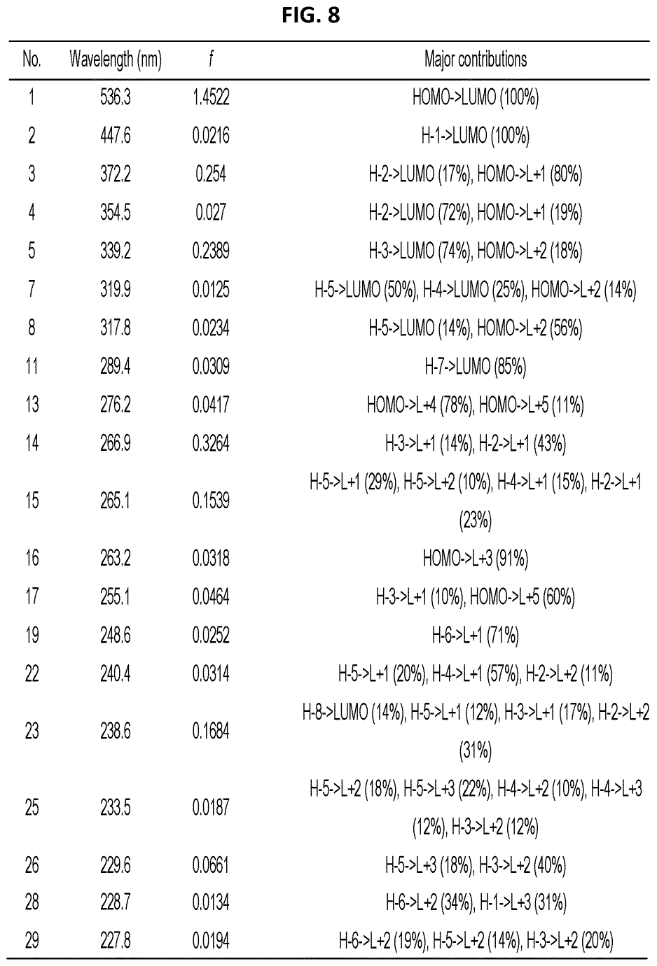

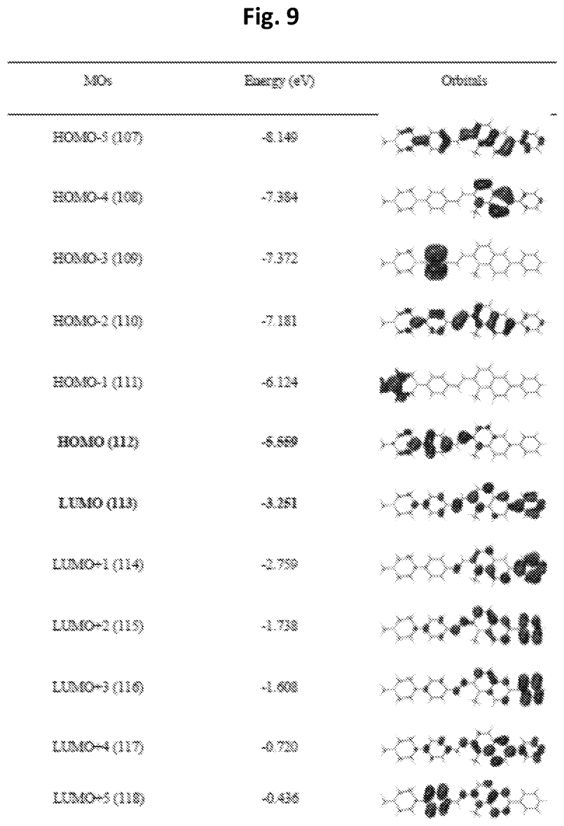

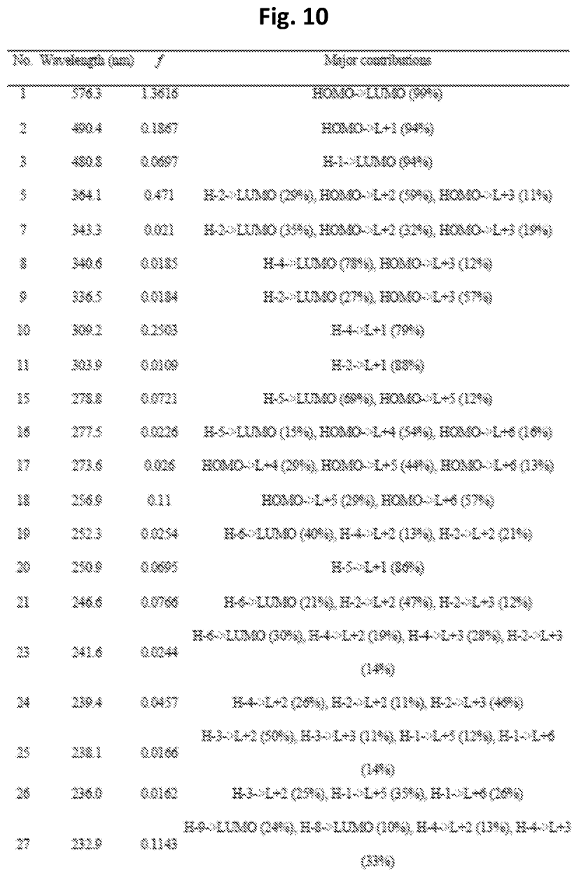

To examine the pH-dependent photophysical properties of PyQPMe, density functional theory (DFT) calculations on PyQPMe were performed (FIGS. 7 and 8) and protonated PyQPMe (protonation at the pyridine unit, FIGS. 9 and 10). As shown in FIG. 2D, the highest occupied molecular orbitals (HOMOs) of both compounds are located at their electron-donating phenyl piperazine side, as expected. The lowest unoccupied molecular orbital (LUMO) of PyQPMe is primarily centered on the methylated quinolinium moiety. In sharp contrast, the LUMO of the protonated PyQPMe has a much wider electron density distribution towards the terminal protonated pyridine, in agreement with its increased positive charge after protonation. Therefore, even though their HOMOs have similar energies (−5.50 and −5.56 eV), the LUMO of the protonated PyQPMe (−3.25 eV) is 0.25 eV lower in energy than that of PyQPMe (−3.00 eV). With such a significant deviation in energy and distribution of the LUMOs from PyQPMe to the protonated PyQPMe, it is thought that the lowest singlet excited state (S₁, primarily from HOMO to LUMO transition) of PyQPMe will show a red shift accordingly after protonation. Indeed, the S₁ states of PyQPMe before and after protonation were computed to be located at 536.3 and 576.3 nm, respectively. Furthermore, the calculated electron density difference maps (EDDMs) of their S₁ states vividly elaborate the change of electron density, where yellow and red imply increase and decrease in electron density, respectively (FIG. 2D).

### Characterization of PyQPMe in Living Cells

In some aspects, the probes of the present disclosure can be administered to living cells, either in vitro or in vivo. To take advantage of the absorption and emission spectra of PyQPMe, this probe was administered in high-resolution microscopy for living cells. The toxicity of PyQPMe was examined at first before characterizing its imaging properties. Cell Counting Kit-8 (CCK-8) was used to investigate the viability of HeLa cells stained with PyQPMe. PyQPMe exhibited no effects on the viability of HeLa cells within 24 h incubation at concentrations of 0~10 μM. The probe's high hydrophilicity (n-octanol/water partition coefficient (Log P)~−0.65) indicates that it enters the cell through endocytosis instead of penetrating freely through cell membranes. Next, PyQPMe was characterized by Leica Stellaris Confocal Microscope in the LIGHTNING module for high-resolution imaging. Under an excitation of 405 nm imaging channel and an incubation time of 10 min, it was found PyQPMe that entered HeLa cells and showed punctate fluorescence signals in the cytoplasm. The signal tended to be endolysosomes because endolysosomes have a low pH range from 4.7 to 6.3. When treating HeLa cells with a high concentration (2 μM) of PyQPMe, the same fluorescence signal inside HeLa cells could be observed, identifying the intake of PyQPMe is through endocytosis. Additionally, it was noted that a significant reduction in fluorescence occurred following treatment with the endocytic inhibitors, chloroquine and NH₄Cl, further identifying that the process of endocytosis is accountable for the uptake of PyQPMe. Since low temperatures can inhibit endocytosis, experiments at a lower temperature were performed. Fewer endolysosomes were detected in cells incubated at a temperature of 4° C. than 37° C., further confirming that the uptake of PyQPMe is an ATP-dependent endocytosis pathway.

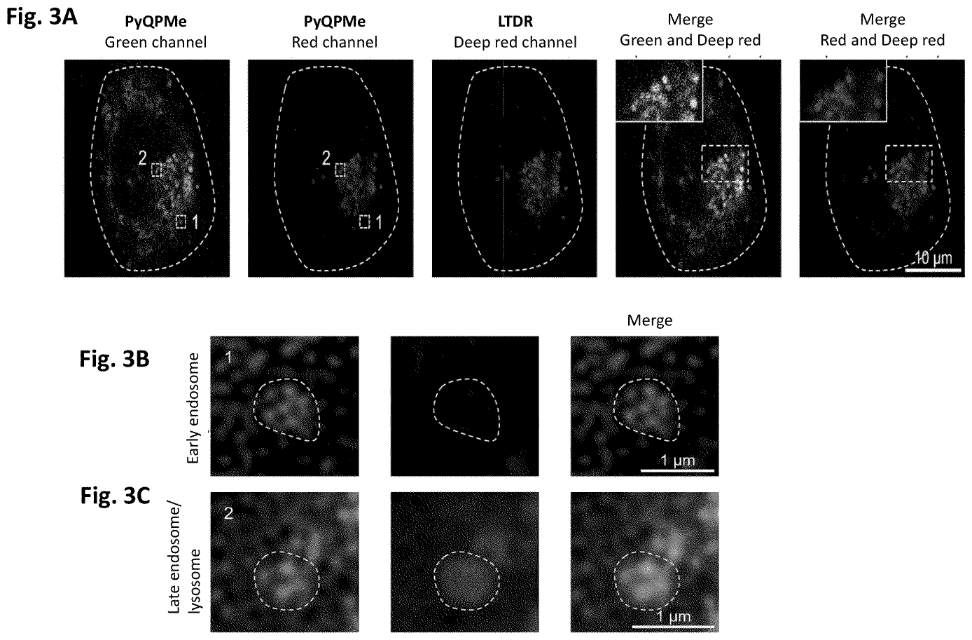

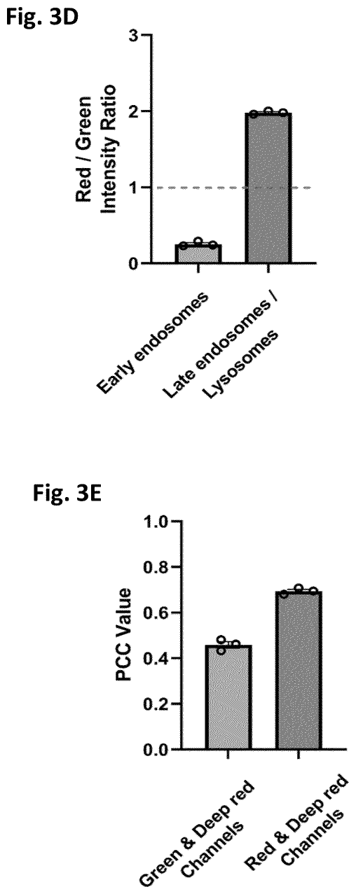

Next, imaging studies of PyQPMe were performed under a dual channel mode with the same excitation of 405 nm (green channel, emission range of 470-530 nm; red channel, emission range of 610-680 nm). Due to the strong green emission properties of PyQPMe over a wide pH range, many puncta fluorescence appeared throughout the cell in the green channel image (FIG. 3A). While in the red channel image, there is less puncta fluorescence in the cell because PyQPMe only emits red light at pH values below 6. Therefore, this imaging property of PyQPMe at different channels can guide one to track different stages of endocytosis. Images were randomly zoomed in and their fluorescence intensity was compared. As shown in FIG. 3B, 3D, early endosomes exhibited clear green fluorescence with a very low ratio of red to green fluorescence intensity. In contrast, due to the low pH value in late endosomes/lysosomes, PyQPMe displayed both green and red fluorescence, and the ratio of red to green intensity was higher than 1 (FIG. 3C, 3D), in full agreement with previous photophysical expectations. Further, the average diameter of PyQPMe red spots is around 0.6 μm, which is in line with the reported size of lysosomes. To further verify the targeting ability of PyQPMe, the cells were co-stained with commercial lysosome dye Lyso-Tracker Deep Red (LTDR) and imaged under a deep red channel (excitation of 640 nm, emission range of 690-740 nm). The results showed that the red channel puncta of PyQPMe overlaid well with the deep red channel puncta of LTDR, and the Pearson correlation coefficient (PCC) value was about 0.70 (FIG. 3A, 3E). While the green channel particles of PyQPMe showed less overlap with the deep red channel of LTDR, the PCC value was 0.46, confirming the presence of early endosomes with a higher pH than lysosomes in the green channel images of PyQPMe.

All the above imaging results demonstrate that the terminal pyridine group of PyQPMe is gradually protonated in the cell through the occurrence of endocytosis, resulting in a red shift of fluorescence. Therefore, due to the different pH values of endolysosomes at different stages, PyQPMe stains total endolysosomes and late endosomes/lysosomes with green and red fluorescence, respectively.

### Characterization of Imaging Properties of PyQPMe-Stained Endolysosomes

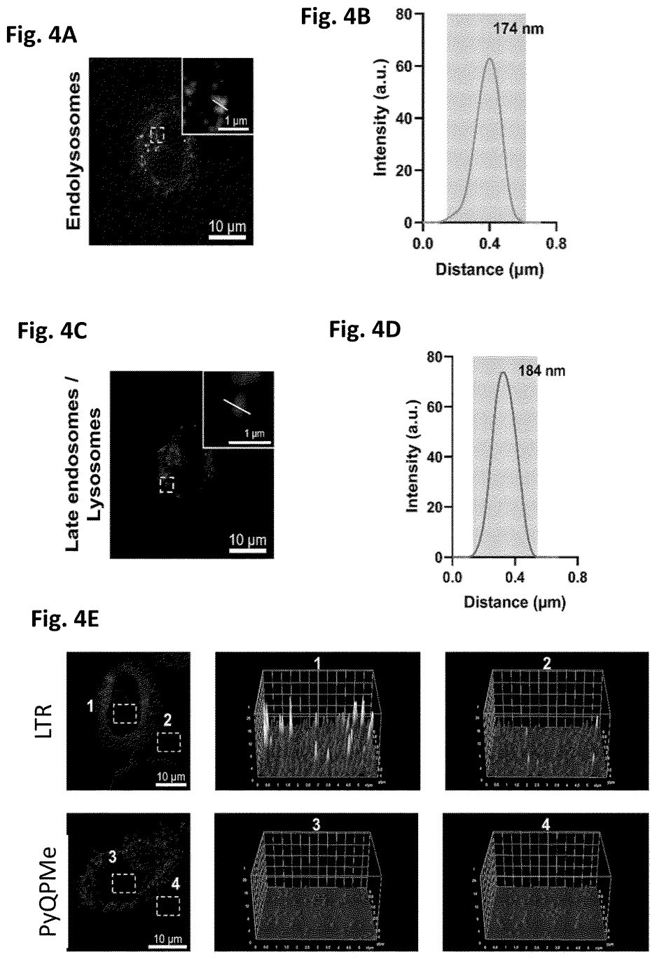

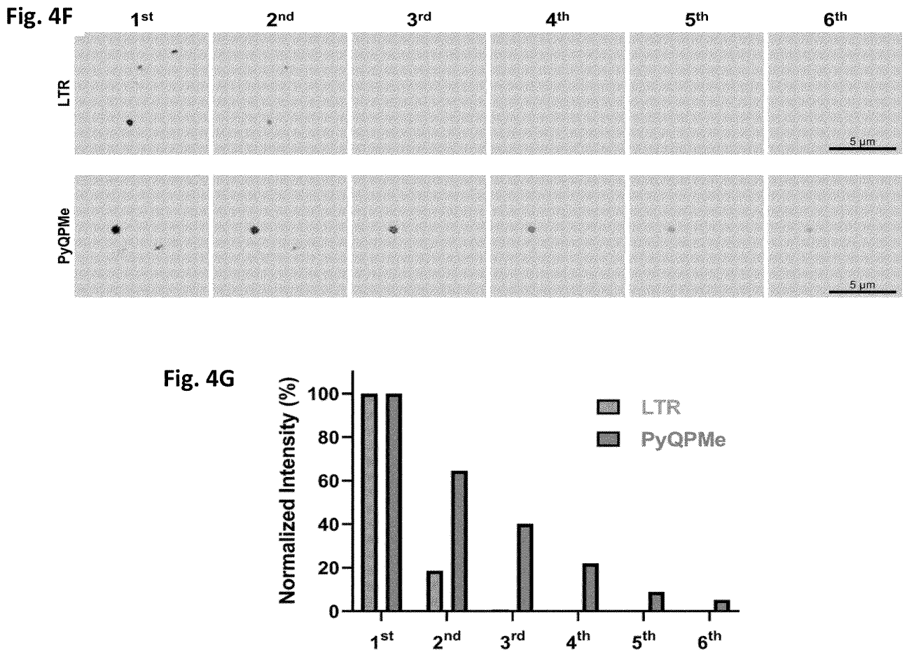

Based on the above photophysical properties, PyQPMe can expected to have superior imaging performance. The fluorescence images of PyQPMe were compared before and after the LIGHTNING module. The confocal images deconvolved by the LIGHTNING module showed better imaging quality, greater precision, higher resolution, and less background fluorescence than those of raw confocal images. To test the resolution limit of PyQPMe, confocal images were stochastically selected, and underlined the areas with luminescence in the images (FIG. 4A, 4C). The FWHMs (full width at half maximum) of these bright puncta fluorescence of PyQPMe-stained endolysosomes and later endosomes/lysosomes are down to 174 nm and 184 nm (FIG. 4B, 4D), respectively.

Since PyQPMe has a large Stokes shift and strong fluorescence intensity, it can effectively reduce the background noise caused by excitation light and microenvironments. To illustrate this merit, the commercial lysosome dye (LysoTracker Red, LTR) was selected for comparison. Two different regions of the background were randomly chosen in each fluorescence image and plotted their 3D fluorescence intensity distribution maps, as shown in FIG. 4E. The results showed that under the same imaging conditions, the background fluorescence of the confocal images of PyQPMe was significantly reduced, and the signal-to-noise ratio was better than that of LTR. Hence, the low background noise is an advantage of PyQPMe for studying the hyperfine structures of cells.

Resistance to photobleaching is a crucial property of probes that determines whether they can be used for long-term fluorescence tracking. To characterize this property, a photobleaching comparison study between LTR and PyQPMe was done in HeLa cells (FIG. 4F). When exposed to continuous confocal laser at 100% power, the fluorescence intensity of LTR disappeared rapidly after two scans, while the fluorescence signal of PyQPMe maintained at 22% of its original value after 40 s of scans (FIG. 4G), thereby demonstrating that PyQPMe has better resistance to photobleaching than the commercial dye. Taken together, these imaging properties of PyQPMe demonstrate that it is a promising probe for high-resolution imaging.

### Characterization of the Dynamics of PyQPMe-Stained Endolysosomes

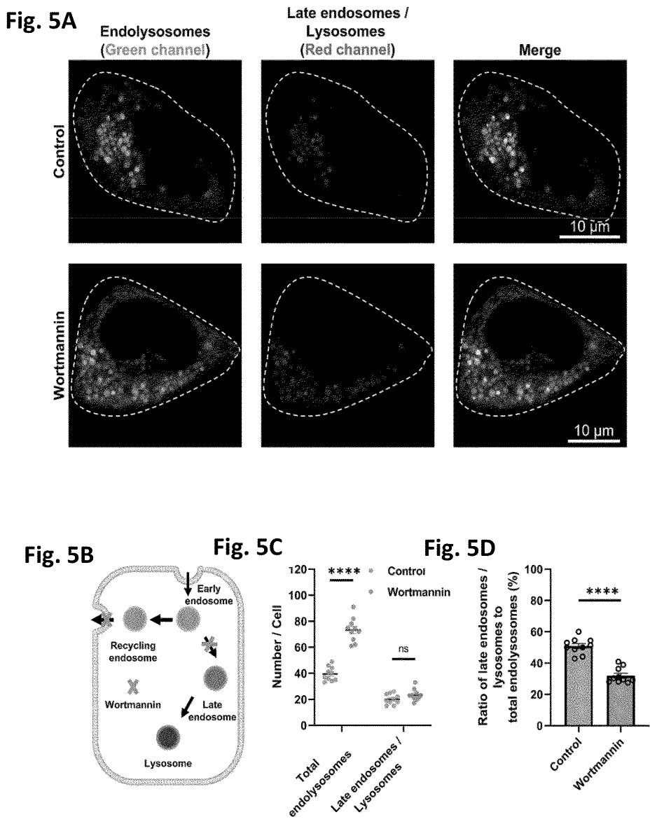

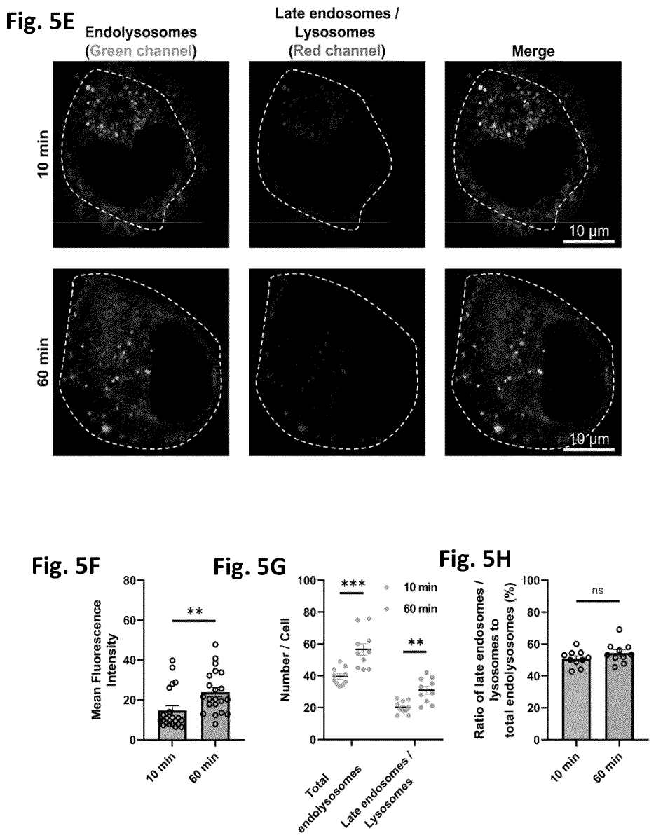

Because of the popularity of quantitative cell biology, it is important to design powerful tools that can digitize individual cells and subcellular organelles to elucidate biological problems in detail. With its great pH response, long-term photostability, and excellent signal-to-noise ratio for high-resolution imaging, PyQPMe was successfully used to track the dynamics of endolysosomes within cells by counting the number of puncta fluorescence signals in both green and red channels (FIG. 5A). First, wortmannin, a protein inhibitor of phosphoinositide 3-kinases (PI3Ks), was used to inhibit early endosomes from maturating into late endosomes (FIG. 5B).⁴⁸⁻⁵⁰ After blocking endocytosis by wortmannin treatment for 24 h, the number of endolysosomes was much higher than that in untreated cells (FIGS. 5A, 5C). Besides, the percentage of late endosomes/lysosomes in total endolysosomes was significantly decreased in cells pretreated with wortmannin (FIG. 5D), further confirming that wortmannin inhibits maturation from early endosomes into late endosomes and recycles more endosomes within the cells.

Next, the effects of different incubation time on the endocytic pathway were tested. HeLa cells were imaged in green and red channels after 10 min and 60 min incubation with PyQPMe, as shown in FIG. 5E. First, with the conversion from early endosomes to late endosomes/lysosomes, the intensity of puncta fluorescence in the red channel increased during 60 min incubation (FIG. 5F), hence demonstrating that this high intensity of dots is due to the low pH of late endosomes and lysosomes. Second, more PyQPMe-stained total endolysosomes and late endosomes/lysosomes were simultaneously detected in HeLa cells incubated for 60 min when compared to the number of endolysosomes in PyQPMe-stained cells incubated for 10 min (FIG. 5G). The percentage of late endosomes/lysosomes in total endolysosomes remained stable regardless of incubation time, as exhibited in FIG. 5H. These results demonstrate that PyQPMe can be effectively used to digitize and track endocytosis to explain the underlying biological mechanisms.

### Dynamics of PyQPMe-Stained Endolysosomes During Autophagy

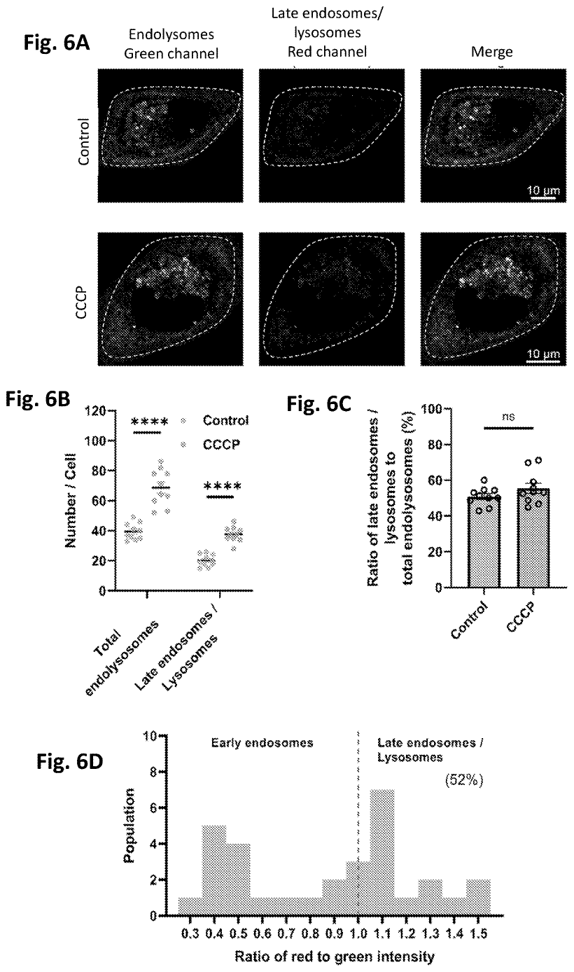

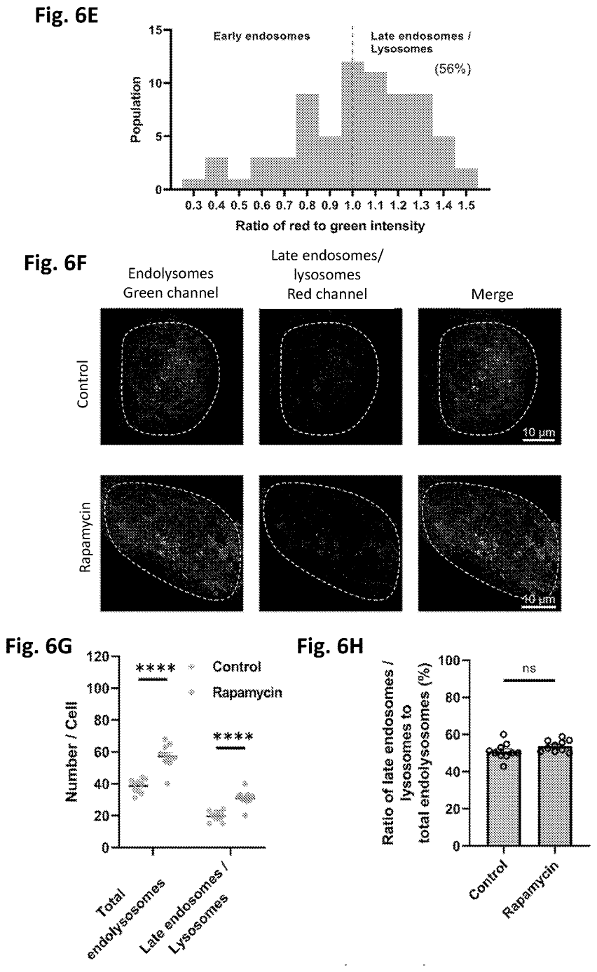

Autophagy is a natural degradation process of cells that removes unnecessary or damaged biological macromolecules or organelles to maintain homeostasis. After autophagosomes engulf unnecessary components, both late endosomes and lysosomes can fuse with autophagosomes to degrade autophagosomal cargo by forming amphisomes and autolysosomes, respectively. To study the behavior of endolysosomes during autophagy, carbonyl cyanide m-chlorophenyl hydrazone (CCCP), a common inducer for mitophagy (one type of autophagy), was exploited to induce autophagy in PyQPMe-stained cells. Cells were treated with 10 μM CCCP for 24 h prior to PyQPMe staining. A significant increase in the number of total endolysosomes and late endosomes/lysosomes was observed in CCCP-treated HeLa cells compared with controls (FIGS. 6A, 6B). The ratio of late endosomes/lysosomes to total endolysosomes remained stable (~55%) during the mitophagy process (FIG. 6C). To further prove this phenomenon, fluorescence intensity of PyQPMe-stained endolysosomes were compared in green and red channel images. As mentioned above (FIG. 2C), pH 5-6 is the cut-off point for the red to green fluorescence intensity ratio of PyQPMe, which perfectly matches the pH changes during conversion from early endosomes to late endosomes. Hence, it can be briefly stated that a population of early endosomes is indicated when the ratio of red to green fluorescence intensity is less than 1. Inversely, when the ratio is greater than 1, it represents the population of late endosomes/lysosomes (FIGS. 3D, 6D, 6E). As shown above, it was first found that the population of both early endosomes and late endosomes increased after CCCP treatment. Then, with quantification of the ratio of red to green intensity, it was identified that both early and late endosomes became more acidic during mitophagy, as revealed by rightward shifts among their population distributions. Furthermore, the ratio of late endosomes/lysosomes to total endolysosomes remained similar for HeLa cells treated with (56%, FIG. 6E) or without (52%, FIG. 6D) CCCP, which is consistent with the results in FIG. 6C.

Rapamycin, a classical autophagy inducer, activates autophagy by inhibiting mTOR protein activation. To investigate the effect of rapamycin on endolysosomes, we treated HeLa cells with 0.5 μM rapamycin for 24 h prior to PyQPMe staining. As shown in FIG. 6F, the rapamycin-treated cells showed more fluorescent spots in both green and red channels. To track the dynamics of endolysosomes in classical autophagy, the total endolysosomes and late endosomes/lysosomes in rapamycin-treated cells were quantified. The results showed that the number of total endolysosomes and late endosomes/lysosomes significantly increased in rapamycin-treated HeLa cells (FIG. 6G). Meanwhile, similar to the CCCP-induced mitophagy (FIGS. 6A, 6C), the proportion of late endosomes/lysosomes to total endolysosomes remained the same (~54%) during classical autophagy process (FIG. 6H). Taken together, these demonstrate that the increased number of lysosomes during autophagy is caused by the augmentation of total endolysosomes, which is reflected in the constant conversion from early endosomes into late endosomes/lysosomes.

Herein, a novel fluorescent probe PyQPMe is presented and described which possesses excellent pH-sensitive photophysical properties. This probe gives a low background noise for high-resolution imaging due to its large Stokes shift and strong fluorescence intensity. To date, fluorescent probes for targeting either lysosomes or late endosomes have been developed. However, suitable probes available to track endolysosomes of different states simultaneously have not existed. Here, the small-molecule probes disclosed herein provide for the simultaneous monitoring of two fluorescence signals in endolysosomes at different stages of interest and minimize the interference from various microenvironments. To sum up, this pH-sensitive probe adds a new powerful tool for the field of endolysosome-related diseases research and contributes to the development of potential drugs targeting dysfunctional endolysosome dynamics in humans. Further, the application of PyQPMe together with high-resolution imaging is useful in determining where increased lysosomes come from during autophagy. The data herein evidence that the increase in the number of lysosomes during autophagy is caused by the augmentation of total endolysosomes; and the conversion rate from early endosomes into late endosomes/lysosomes remains constant. This result implies a complicated protein system governing the dynamics of endolysosomes, which requires a precise cooperation of multiple proteins.

### Examples

### Synthesis of PyQPMe

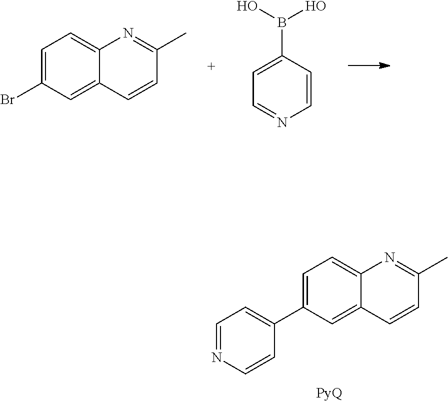 [SMILES: Cc1ccc2cc(Br)ccc2n1 ; OB(O)c1ccncc1 ; Cc1ccc2cc(-c3ccncc3)ccc2n1]

Synthesis of 2-methyl-6-(pyridin-4-yl)quinoline (PyQ). To a three-neck round flask, 5.0 mmol (1.11 g) 6-bromo-2-methylquinoline, 1.5 equiv. (183 mg) pyridin-4-ylboronic acid, 0.1 equiv. Pd(PPh₃)₄, 3.0 equiv. K₂CO₃, and 0.1 equiv. N(Bu)₄Br were added under N₂, then 180 mL toluene, 120 mL EtOH, and 60 mL H₂O were added. The reaction mixture was stirred at 100° C. overnight. When the reaction finished, the mixture was diluted with EtOAc and washed with water. The combined organic phase was concentrated. The pure product was obtained through chromatography on silica using EtOAc, EtOH, and hexanes as the eluent. Eventually, 0.84 g target product was obtained with a yield of 76%. ¹H NMR (400 MHz, CDCl₃) δ 8.70-8.68 (m, 2H), 8.11-8.08 (m, 2H), 8.01 (d, 1H), 7.94-7.91 (m, 1H), 7.60-7.59 (m, 2H), 7.33 (d, 1H), 2.76 (s, 3H). ¹³C NMR (100 MHz, CDCl₃) δ 160.22, 150.54, 148.10, 147.77, 136.66, 135.42, 129.79, 128.28, 126.69, 126.09, 122.95, 121.92, 25.63.

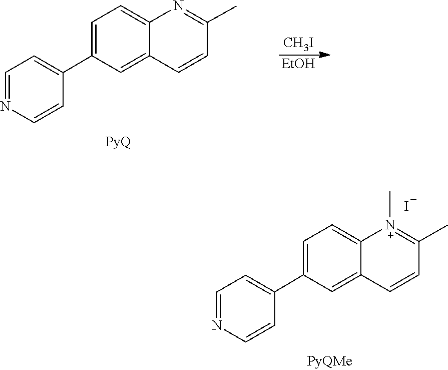 [SMILES: "EtOH" ; Cc1ccc2cc(-c3ccncc3)ccc2n1 ; "CH3I" ; Cc1ccc2cc(-c3ccncc3)ccc2[n+]1C ; [I-]]

Synthesis of 1,2-dimethyl-6-(pyridin-4-yl)quinolin-1-ium (PyQMe). To a 25 mL round flask, 0.5 mmol (110 mg) PyQ and 2.0 equiv. CH₃I were dissolved in 10 mL dry EtOH. The reaction mixture was stirred at 80° C. overnight. When the reaction was finished, light yellow precipitates formed in the solution. After filtered and washed with cool ethanol and dimethyl ether. The pure product was obtained through recrystallization from methanol. After drying, the target product was obtained in a nearly quantitative yield (190 mg). ¹H NMR (400 MHz, D₂O) δ 8.59 (d, 2H), 8.02 (m, 2H), 7.98-7.94 (m, 2H), 7.77-7.74 (m, 1H), 7.55 (d, 1H), 7.25 (d, 1H), 4.27 (s, 3H), 2.50 (s, 3H). ¹³C NMR (100 MHz, D₂O) δ 162.03, 154.08, 146.97, 144.69, 138.22, 129.98, 128.43, 127.81, 127.58, 125.87, 124.28, 123.53, 47.26, 23.88.

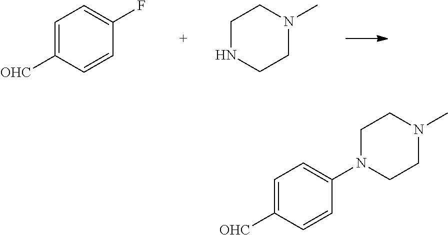

Synthesis of 4-(4-methylpiperazin-1-yl)benzaldehyde. 10 mmol p-fluorobenzaldehyde (1.24 g), 1.1 equiv. 1-methylpiperazine (1.10 g), 2.0 equiv. K₂CO₃ (2.76 g), and DMF (100 mL) were added to a round flask. The mixture was stirred at 100° C. for 16 h under N₂. After the reaction was completed, the mixture was poured into cool water and the precipitation was formed. The precipitation was filtered and washed with cool water. After dried at room temperature, the pure product was obtained (1.98 g) through chromatography on silica with EtOAc and hexanes as the eluent (reaction yield 97%). ¹H NMR (400 MHz, CDCl₃) δ 9.76 (s, 1H), 7.74 (d, 2H), 6.90 (d, 2H), 3.40 (t, 4H), 2.54 (t, 4H), 2.34 (s, 3H). ¹³C NMR (100 MHz, CDCl₃) δ 190.52, 155.11, 131.95, 127.21, 113.64, 54.78, 47.16, 46.22.

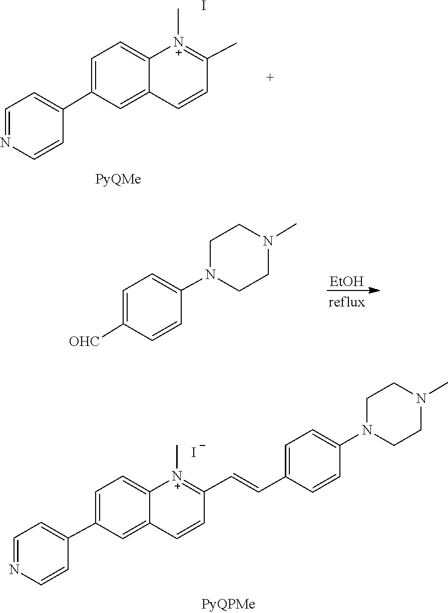

Synthesis of (E)-1-methyl-2-(4-(4-methylpiperazin-1-yl)styryl)-6-(pyridin-4-yl)quinolin-1-ium (PyQPMe). To a round bottom flask, 0.1 mmol PyQMe (36.5 mg) and 1.1 equiv. 4-(4-methylpiperazin-1-yl) benzaldehyde (22.4 mg) were dissolved in 10 mL anhydrous ethanol. The solution was refluxed for 48 h under N₂. After cooling to room temperature, the solvent was evaporated under reduced pressure. The residue was purified by washing with cool ethanol, EtOAc, hexanes, and dimethyl ether. Finally, 6.8 mg desired product was obtained in a yield 12%. ¹H NMR (400 MHz, DMSO) δ 9.06 (d, 2H), 8.76 (d, 1H), 8.64 (d, 2H), 8.42 (d, 1H), 8.38-8.35 (m, 1H), 8.13 (d, 1H), 7.93 (d, 1H), 7.85 (d, 1H), 7.61 (d, 2H), 7.31 (d, 1H), 7.10 (d, 1H), 4.35 (s, 3H), 3.25 (m, 4H), 2.45 (m, 4H), 2.33 (s, 3H). ¹³C NMR (100 MHz, DMSO) δ 158.29, 153.55, 149.94, 148.98, 145.64, 137.40, 135.58, 130.33, 19.77, 128.96, 128.79, 128.20, 127.35, 126.79, 125.23, 124.10, 121.14, 115.57, 52.21, 47.10, 44.96.42.21.

### Materials and Methods

All commercially available reagents were purchased and used without further treatment, including 6-bromo-2-methylquinoline, pyridin-4-ylboronic acid, p-fluorobenzaldehyde, 1-methylpiperazine. Carbonyl cyanide m-chlorophenylhydrazone (CCCP, #C2759) was purchased from Sigma. LysoTracker Red DND-99 (LTR, #L7528) and LysoTracker Deep Red (LTDR, #L12492) were purchased from Invitrogen (Thermo Fisher Scientific). All fluorescent dyes were used under the manuals. Phosphate-buffered saline solution (PBS, #SH30256.01) was purchased from Hyclone (GE Healthcare Life Sciences). Dulbecco's modified Eagle's medium (DMEM, #11965092), bovine serum (FBS, #26140079), and Penicillin-streptomycin were purchased from Gibco (Thermo Fisher Scientific, USA).

Characterization of PyQPMe. The ¹H and ¹³C NMR spectra were recorded on a Bruker Avance III HD Ascend 400 MHz NMR spectrometer. Chemical shifts for protons are referenced to the residual solvent peak (CDCl3, ¹H NMR: 7.26 ppm; D2O, ¹H NMR: 4.79 ppm; d₆-DMSO, ¹H NMR: 2.50 ppm), while chemical shifts for carbons are referenced to the residual solvent peaks (CDCl₃, ¹³C NMR: 77.16 ppm, d₆-DMSO; ¹³C NMR: 39.51 ppm). The following abbreviations (or combinations thereof) were used to explain multiplicities: s=singlet, d=doublet, m=multiplet. Matrix-Assisted Laser Desorption Ionization (MALDI) mass spectrometry was performed on a Bruker Biflex III MALDI-TOFMS instrument.

Phosphate buffer solution. A series of standard pH buffer solutions were prepared by mixing 0.2 M Na₂HPO₄·7H₂O, 0.2 M KH₂PO₄, and 0.2 M H₃PO₄ with varied volume ratios, and the pH values were measured by using a Mettler Toledo pH-meter.

Absorption and emission measurements. 3.0 mL phosphate buffer solution and 6.44 μL probe stock solution (4.66 mM) were mixed well, and an appropriate portion of the solution was transferred to a quartz cell with 1 cm optical length to collect the absorption spectra on an Agilent Cary 453 spectrophotometer. The fluorescence spectra were recorded on a HORIBA Fluorolog QM spectrofluorometer.

Fluorescence reversibility of PyQPMe. The pH of a PyQPMe solution between pH 3.0 and pH 8.5 was adjusted back and forth by adding 2.0 M KOH or 3.0 M HCl, which was monitored by a Mettler Toledo pH meter.

Computational methods. All calculations were performed with the Gaussian 16 W program package employing the DFT method with Becke's three-parameter hybrid functional and Lee-Yang-Parr's gradient corrected correlation functional (B3LYP)60-62. 6-31G* basis set was applied for H, C, O, and N. The geometries of the singlet ground states of compounds were optimized in H2O using the conductive polarizable continuum model (CPCM). The local minimum on each potential energy surface was confirmed by frequency analysis. Time-dependent DFT calculations produced the singlet excited states of each compound starting from the optimized geometry of the corresponding singlet ground state, using the CPCM method with H₂O as the solvent. The calculated absorption spectra, electronic transition contributions, and electron density difference maps (EDDMs) were generated by GaussSum 3.0.63 The electronic orbitals were visualized using VMD 1.9.4a51.64.

The measurements of hydrophilicity. Hydrophilicity was determined by a shake-flask ultraviolet spectrophotometry method to measure the n-octanol/water partition coefficients (Log P). First, 25 mL of n-octanol and 25 mL of water were mixed at room temperature. After 24 h, the standard solutions of the sample (10 μM) were prepared by using the preceding saturated n-octanol phase and water phase solution, respectively. Later, the test solutions were prepared by mixing 10 ml of standard solution in n-octanol and 10 ml of standard solution in water. After shaking, the mixture of test solution was centrifuged for 5 min. The n-octanol phase layer was separated from the water phase layer and the absorbance of the two layers was measured by Agilent Cary 8453 spectrophotometer. Based on the Beer-Lambert law, the concentrations of the sample in the n-octanol phase (Co) and the water phase (Cw) were calculated. Finally, the n-octanol/water partition coefficients (Log P) were further calculated by using the following equation:

$$Log P = Log ( Co / Cw ) .$$

Cell culture. HeLa cells and PC12 cells were gifted. Cells were cultured in Dulbecco's modified Eagle medium supplemented with 10% FBS (fetal bovine serum, Gibco BRL), 100 μg/mL streptomycin (Gibco BRL), and 100 U/mL penicillin (Gibco BRL). The cells were cultured at 37° C. with 5% CO₂ in a humidified incubator.

Leica Stellaris 8 Confocal imaging. High-resolution images were acquired on a commercial Leica Stellaris 8 Confocal Microscope under LIGHTNING mode. Laser powers were set at 10% intensity for 405 nm and 640 nm channels. The objective was 63×/1.4. High-resolution images were captured from the randomly selected field of view and analyzed with Leica LAS X and ImageJ software. All the images were exported on the Auto scale of the Leica LAS X software. Cells were seeded on glassed-bottomed cell dishes (MatTek) for 24 hours. Cells stained with PyQPMe (20 nM, 10 min) were treated with Wortmannin (1 μM, 24 h), CCCP (10 PM, 24 h), rapamycin (0.5 μM, 24 h), chloroquine (25 μM, 30 min), and NH₄Cl (50 mM, 30 min). All PCC values were analyzed in the CellProfiler.

Cell viability and cytotoxicity assay. HeLa cells were seeded in a 96-well plate at a density of 1×104 cells per well in 100 μL culture medium with different concentrations of PyQPMe at a given time. The viability was determined by adding 10 μL reagent from a cell counting kit-8 (CCK-8) (Dojindo Molecular Technologies, Inc.) and incubating at 37° C. for 1 h. CCK-8 solution was added to each well and the OD value for each well was read at wavelength 490 nm on a microplate reader (BioTek Instruments, Inc., USA). The interference of absorbance from PyQPMe at 490 nm was minimal compared to the CCK-8 reagent during measurement.

Statistical analysis. Statistics and graphing were performed by using GraphPad Prism 9 and Excel.

Various modifications of the present disclosure, in addition to those shown and described herein, will be apparent to those skilled in the art of the above description. Such modifications are also intended to fall within the scope of the appended claims.

It is appreciated that all reagents are obtainable by sources known in the art unless otherwise specified.

It is also to be understood that this disclosure is not limited to the specific aspects and methods described herein, as specific components and/or conditions may, of course, vary. Furthermore, the terminology used herein is used only for the purpose of describing particular aspects of the present disclosure and is not intended to be limiting in any way. It will be also understood that, although the terms “first,” “second,” “third” etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, “a first element,” “component,” “region,” “layer,” or “section” discussed below could be termed a second (or other) element, component, region, layer, or section without departing from the teachings herein. Similarly, as used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

Reference is made in detail to exemplary compositions, aspects and methods of the present disclosure, which constitute the best modes of practicing the disclosure presently known to the inventors. The drawings are not necessarily to scale. However, it is to be understood that the disclosed aspects are merely exemplary of the disclosure that may be embodied in various and alternative forms. Therefore, specific details disclosed herein are not to be interpreted as limiting, but merely as a representative basis for any aspect of the disclosure and/or as a representative basis for teaching one skilled in the art to variously employ the present disclosure.

Patents, publications, and applications mentioned in the specification are indicative of the levels of those skilled in the art to which the disclosure pertains. These patents, publications, and applications are incorporated herein by reference to the same extent as if each individual patent, publication, or application was specifically and individually incorporated herein by reference.

The foregoing description is illustrative of particular embodiments of the disclosure, but is not meant to be a limitation upon the practice thereof. The following claims, including all equivalents thereof, are intended to define the scope of the disclosure.

1. A fluorescent probe comprising a molecule comprised of a quinolinium, a pyridine, and a piperazine linked together.

2. The fluorescent probe of claim 1, further comprising a phenyl and a C═C double bond linking the quinolinium and the piperazine.

3. The fluorescent probe of claim 1, wherein the piperazine is methylated.

4. The fluorescent probe of claim 1, wherein the molecule comprises the structure of formula I: 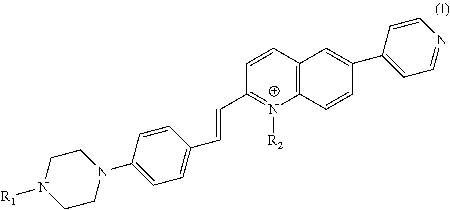
wherein R₁ and R₂ are independently an alkyl of C₁-C₈ in length and wherein the pyridine is optionally protonated.

5. The fluorescent probe of claim 1, wherein the molecule comprises the structure of formula II: 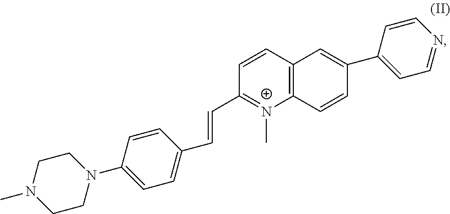 [SMILES: CN1CCN(c2ccc(/C=C/c3ccc4cc(-c5ccncc5)ccc4[n+]3C)cc2)CC1]
wherein the pyridine is optionally protonated.

6. The fluorescent probe of claim 1, further comprising a halide and wherein the fluorescent probe is a salt.

7. A method for monitoring endolysosomes in a cell, comprising administering the fluorescent probe of claim 1 to a cell.

8. The method of claim 7, further comprising viewing the cell at at least one time point and optionally capturing an image of the cell.

9. The method of claim 8, wherein the cell is viewed through a green channel, a red channel, or both.

10. The method of claim 7, wherein the cell is treated with a further compound at a time point prior to, simultaneously, or after administering the probe to the cell.

11. A fluorescent probe comprised of a molecule, wherein the molecule comprises the structure of formula II:

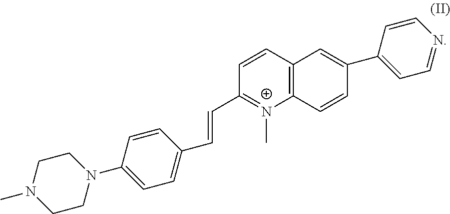 [SMILES: CN1CCN(c2ccc(/C=C/c3ccc4cc(-c5ccncc5)ccc4[n+]3C)cc2)CC1]

12. The fluorescent probe of claim 11, wherein the pyridine is optionally protonated.

13. The fluorescent probe of claim 11, further comprising a halide and wherein the fluorescent probe is a salt.

14. The fluorescent probe of claim 13, wherein the halide is iodide.

15. A method for monitoring endolysosomes in a cell, comprising administering the fluorescent probe of claim 11 to a cell.

16. The method of claim 15, further comprising viewing the cell at at least one time point and optionally capturing an image of the cell.

17. The method of claim 16, wherein the cell is viewed through a green channel, a red channel, or both.

18. The method of claim 15, wherein the cell is treated with a further compound at a time point prior to, simultaneously, or after administering the probe to the cell.
